(19) United States
(12) Patent Application Publication
(10) Pub. No.: US 2012/0302905 A1
(43) Pub. Date: Nov. 29, 2012

(54) METHOD, APPARATUS AND COMPUTER PROGRAM PRODUCT FOR MONITORING PHYSIOLOGICAL SIGNALS

(75) Inventor: Mikko Kaski, Espoo (FI)

(73) Assignee: GENERAL ELECTRIC COMPANY, Schenectady, NY (US)

(21) Appl. No.: 13/117,338

(22) Filed: May 27, 2011

(51) Int. Cl. A61B 5/0402

(52) U.S. Cl. 600/513

(57) ABSTRACT

A method, apparatus, and computer program product for monitoring a physiological signal of a subject are disclosed. To provide a mechanism that allows perception of the general clinical state of the subject easily and without expertise, a property measure is derived from at least one physiological signal obtained from a subject, wherein each property measure is indicative of a predetermined property of a respective physiological signal in a time window, and wherein the deriving is performed in consecutive time windows, thereby to obtain at least one property measure sequence. At least one indication signal is produced to be presented to a user, wherein the producing comprises determining signal attributes for the at least one indication signal based on the at least one property measure sequence, and the at least one indication signal is presented to the user.

### BACKGROUND OF THE INVENTION

This disclosure relates generally to patient monitors. More particularly, the present invention relates to monitoring of physiological signals, especially electrocardiograms, in patient monitors.

Patient monitors are electronic devices designed to display physiological information about a subject. Electrocardiogram (ECG), electroencephalogram (EEG), plethysmographic signals, and signals related to blood pressure, temperature, and respiration represent typical physiological information contained in full-size patient monitors. Patient monitors are typically also furnished with alarming functionality to alert the nursing staff when a vital sign or physiological parameter of a patient exceeds or drops below a preset limit. Alarms are normally both audible and visual effects aiming to alert the staff to a life-threatening condition or to another event considered vital. In most monitors, the alarm limits may be defined by the user, since the limits typically depend on patient etiology, age, gender, medication, and various other subjective factors. Each specific physiological parameter, such as heart rate or blood pressure, may also be assigned more than one alarm limit. Furthermore, there is a lot of data trended and available for caregivers to be reviewed in a patient monitor. However, this data is typically numeric and needs additional analyses to be useful for the user. Alarms are often activated in a phase where the situation is already critical and a vast amount of trended data is available in a form that needs further processing to be useful. Current patient monitors cannot process this data quickly enough to a form that would be directly useful for caregivers to take an action in advance in order to avoid a critical situation

For recording an electrocardiogram, electrocardiographic leads are used at specified locations of the subject for recording ECG waveforms. In typical clinical practice, 12 leads are used to the record the ECG. However, the number of leads used may vary. Each lead records a waveform representing the electrical activity generated by the heart cardiac cycle by cycle and together the lead recordings provide spatial information about the heart's electrical activity.

A normal cardiac cycle includes contractions of the atrial muscles, which are activated by the autonomic sinoatrial node (SA node), also called the sinus node. An electrophysiologic (EP) signal generated by the SA node spreads in the right and left atrium leading to their contraction. The EP signal further reaches the atrioventricular node (AV node) situated between the atria and the ventricles. The AV node delays the EP signal, giving the atria time to contract completely before the ventricles are stimulated. After the delay in the AV node, the EP signal spreads to the ventricles via the fibers of the His-Purkinje system leading to the contraction of the ventricles. After the contraction, the atria are relaxed and filled by blood coming from venous return. The entire cardiac cycle is the combination of atrial and ventricular contraction, i.e. depolarization, and their relaxation, i.e. repolarization.

In this connection, reference is made to FIGS. 1 and 2, which show 8 different lead signals: I, II, and V1-V6. FIG. 1 illustrates an example of a normal ECG, while FIG. 2 illustrates an example of an abnormal ECG. When a physician analyzes an ECG of a subject, i.e. a set of ECG lead signals as shown in FIGS. 1 and 2, (s)he normally follows a standardized sequence of steps to avoid missing any abnormalities in the cardiac function. These steps typically include measurements that are usually made in the frontal plane leads as well as rhythm, conduction, and waveform analyses.

In a clinical environment, the first decision needed normally in view of an ECG is whether or not the ECG is normal. For a trained physician the examination of an ECG in this respect is more or less a routine task. For example, from FIG. 1 a trained physician may easily see that the heart axis is normal and V lead progression is also normal, whereas the example of FIG. 2 indicates abnormalities both in terms of heart axis and V lead progression. However, in a clinical environment a trained physician is not always available for ECG interpretation and nurses are not normally trained to analyze the ECG waveforms. Current patient monitors lack intelligence to evaluate the ECG waveforms in this respect and cannot therefore assist the nurses in the decision-making. Therefore, physicians may be called for unnecessarily or abnormalities in cardiac function may remain unnoticed before a trained physician is available for ECG analysis.

Furthermore, in a clinical environment a need often arises to examine the history data, such as ECG history data, of the patient, thereby to see if similar events have occurred in the past. In addition, it is also important to know if the current signal is relatively different to what it was earlier. That is, a patient monitor should be able to indicate significant changes in the data with a mechanism from which a caregiver may intuitively grasp that a significant change has occurred. However, current patient monitors lack such a simple and efficient tool for quickly browsing through the history data to promptly get an impression of the possible occurrences of a specific event in the past and to perceive significant changes as they occur.

### BRIEF DESCRIPTION OF THE INVENTION

The above-mentioned problems are addressed herein which will be comprehended from the following specification. In the disclosed monitoring system, property measures indicative of the presence/absence of predetermined signal/parameter characteristics are determined based on one or more physiological signals and the property measures obtained are used to determine signal attributes for one or more indication signals, thereby to obtain one or more indication signals that symbolize how well the physiological signal(s)/parameter(s) conform(s) to the predetermined characteristics. In case of an ECG signal, the property measures may be determined based on the QRS waves/complexes, for example. Instead of indicating a certain property of the physiological signal(s), the indication signal may also symbolize the degree of normality of the said signal(s). Due to the easily perceptible attributes of the indication signal(s), the decision on whether or not the physiological signal, such as an ECG, is normal, may be made easily, and specific events may be quickly searched for from past indication signal data.

In an embodiment, a method for monitoring a physiological signal of a subject comprises deriving a property measure from at least one physiological signal obtained from a subject, wherein each property measure is indicative of a predetermined property of a respective physiological signal in a time window, and wherein the deriving is performed in consecutive time windows, thereby to obtain at least one property measure sequence. The method further comprises producing at least one indication signal to be presented to a user, wherein the producing comprises determining signal attributes for the at least one indication signal based on the at least one property measure sequence and presenting the at least one indication signal to the user.

In another embodiment, an apparatus for monitoring a physiological signal of a subject comprises an analysis unit adapted to derive a property measure from at least one physiological signal obtained from a subject, wherein each property measure is indicative of a predetermined property of a respective physiological signal in a time window, and wherein the analysis unit is adapted to derive the property measure in consecutive time windows, thereby to obtain at least one property measure sequence. The apparatus further comprises a presentation unit adapted to determine signal attributes for at least one indication signal based on the at least one property measure sequence, produce the at least one indication signal, and present the at least one indication signal to a user.

In a still further embodiment, a computer program product for monitoring a physiological signal of a subject comprises a first program product portion adapted to derive a property measure from at least one physiological signal obtained from a subject, wherein each property measure is indicative of a predetermined property of a respective physiological signal in a time window, and wherein the first program product portion is adapted to derive the property measure in consecutive time windows, thereby to obtain at least one property measure sequence. The computer program product further comprises a second program product portion adapted to determine signal attributes for at least one indication signal based on the at least one property measure sequence, produce the at least one indication signal, and present the at least one indication signal to a user.

Various other features, objects, and advantages of the invention will be made apparent to those skilled in the art from the following detailed description and accompanying drawings.

### BRIEF DESCRIPTION OF THE DRAWINGS

FIG. 1 illustrates an example of a normal ECG;

FIG. 2 illustrates an example of an abnormal ECG;

FIG. 3 is a flow diagram illustrating an embodiment of the operation of an ECG monitor in terms of presenting ECG lead signal data to a user;

FIG. 4 illustrates an example of determination of a property measure indicative of QRS polarity;

FIGS. 5 to 7 illustrate the mapping of different QRS waves to signal attributes of an indication signal to be presented to a user of the monitoring apparatus;

FIG. 8 illustrates an example of an ECG display presented to a user;

FIG. 9 illustrates another example of an ECG display presented to a user;

FIG. 10 illustrates an embodiment of an ECG monitoring apparatus/system; and

FIG. 11 illustrates the operational entities of the ECG monitoring apparatus/system in terms of the presentation of the indication signal to a user.

### DETAILED DESCRIPTION OF THE INVENTION

As discussed above, a physician/cardiologist normally follows a standardized sequence of steps when analyzing the ECG, thereby to avoid missing any abnormalities in the cardiac function. These steps include certain measurements, like determining heart axis, also termed QRS axis. The axis, which indicates the direction of the average depolarization within the heart, may be determined by examining, for example, the I, AVF, and AVR lead signals. With normal rhythm, both the I and AVF lead signals should indicate a positive direction, while the AVR lead signal should indicate a negative direction. If this is not the case, something may be wrong and further examinations should be carried out. So, the axis may indicate generally that something is wrong, but an abnormal axis cannot indicate what is wrong.

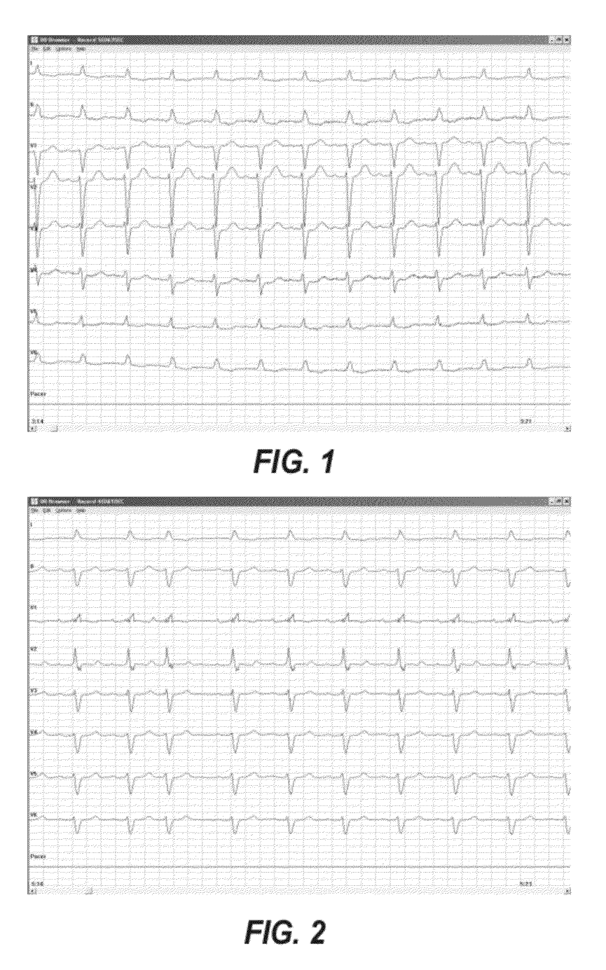

In an embodiment of the disclosed patient monitor, the interpretation rules used by a cardiologist are emulated by determining, for each lead signal and for each cardiac cycle, a property measure that is indicative of the presence/absence of the same ECG feature(s) that the cardiologist checks in the lead signals. Each property measure obtained from an ECG lead signal may be mapped to a signal attribute which is then assigned to an indication signal or signal segment that corresponds to the ECG lead signal, so that the said indication signal or signal segment assumes the attribute in the respective cardiac cycle. Thus, each ECG lead signal is represented by an indication signal or signal segment. The presentation given to the user may be regarded to comprise either one indication signal including a signal segment for each ECG lead signal or several indication signals, one for each ECG lead signal. Below, the examples relate to visualization of the ECG lead signals on a screen of a display unit and it is assumed that the indication signal (video signal) comprises a segment for each ECG lead signal. In the examples below, each indicator signal segment corresponds to one horizontal row on the screen of a display unit, similarly as one ECG lead signal forms one “horizontal row” in the waveform presentation of the ECG lead signals, cf. FIGS. 1 and 2.

In one embodiment, the property measure is indicative of the polarity of the QRS complex/wave, while attributes of indication signal are signal colors that depend on the property measures. That is, the color of the indication signal indicates how well the QRS wave conforms to the polarity feature. The indication signal segments may then be displayed vertically aligned below each other, i.e. similarly as the original ECG lead signals are displayed in FIGS. 1 and 2. Below, this color-based presentation of the ECG lead signals is termed a user display window. As normal ECG corresponds to a specific coloring of the user display window, it is easy for a nurse to see, if a window obtained from a subject deviates from a window presenting a normal ECG.

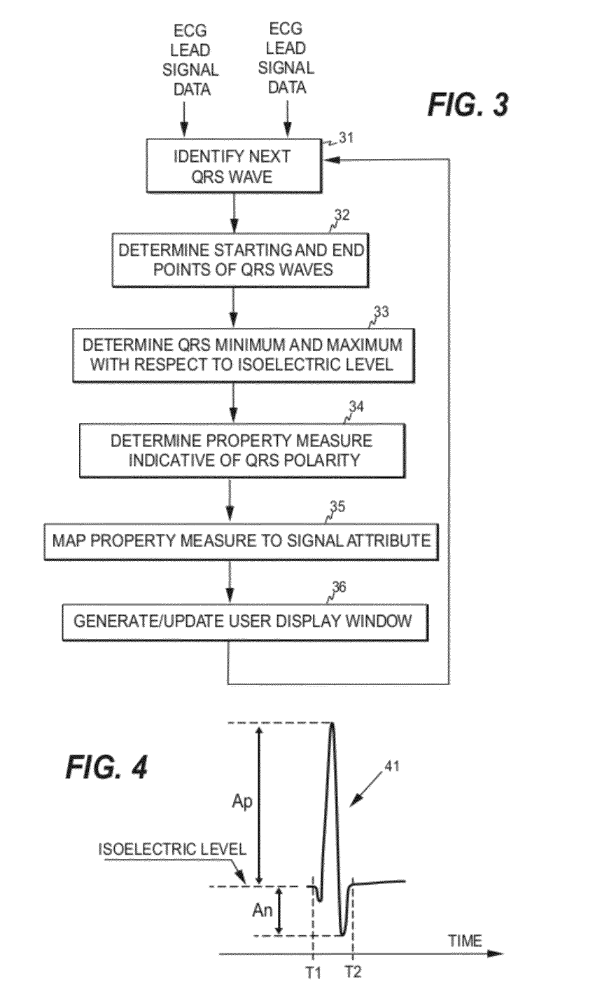

FIG. 3 illustrates one embodiment of the steps carried out to generate the user display window. First, the QRS waves are identified in each lead signal (step 31) and the starting and end points of the QRS waves are determined for each lead signal (step 32). Having determined the temporal position of the current QRS wave for each lead signal, the patient monitor determines QRS maximum and minimum with respect to the isoelectric level (step 33) and calculates a property measure indicative of QRS polarity based on the maximum and minimum (step 34) for each lead signal. Each property measure is then mapped to a signal attribute in step 35. Each signal attribute is assigned to the current time window of a respective indication signal segment, where the time window corresponds to the current cardiac cycle. Thus, in current time window each indication signal segment adopts the respective signal attribute. Here, each indication signal segment corresponds to one horizontal row of the user display window and each segment comprises consecutive time windows that correspond to the consecutive cardiac cycles of the subject. Thus, the number of indication signal segments corresponds to the number of lead signals, i.e. each lead signal is represented by a respective horizontal indication signal segment in the user display window.

The indication signal is then produced and displayed to the user in step 36. The presentation may be similar to the presentation of FIGS. 1 and 2, except that now each ECG lead signal is replaced by the respective indication signal segment. The above steps are repeated for each QRS complex of each lead signal, thereby to be able to update the user display window cardiac cycle-by-cycle. In each cardiac cycle, each indication signal segment adopts a signal attribute that corresponds to the property measure derived from the respective lead signal in that cardiac cycle. The displaying may be started when a predetermined number of cardiac cycles have been examined.

FIG. 4 illustrates an example of steps 33 and 34 by showing an example of one QRS wave 41. In step 32, the starting point T1 and the end point T2 of the QRS wave are determined. In step 33, the maximum positive amplitude Ap and the minimum negative amplitude An are searched for from between T1 and T2. Ap corresponds to QRS maximum, while An corresponds to QRS minimum. In step 34, the values of Ap and An are employed to calculate a property measure PM indicative of QRS polarity. In this example, PM is calculated as follows: PM=100×[abs(Ap)/(abs(Ap)+abs(An))], where abs(x) refers to the absolute value of x. Consequently, PM indicates the percentage of the peak amplitude in the positive direction relative to the sum of the absolute peak amplitudes in the two directions (i.e. positive and negative).

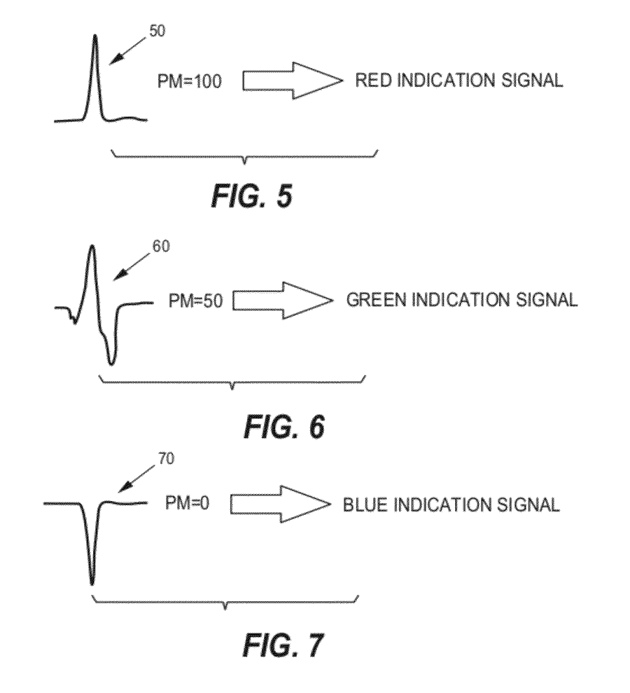

In different ECG leads, the QRS complex may look quite different. FIGS. 5 to 7 illustrate respectively three different QRS waves 50, 60, and 70, and an example of the application of steps 34 and 35 of FIG. 3 on the said QRS waves. In the example of FIG. 5, the QRS wave 50 comprises a positive peak only, i.e. An=0, and therefore the property measure PM obtains a value of 100 in step 34. In the example of FIG. 6, the QRS wave 60 comprises positive and negative peaks with substantially equal amplitudes, i.e. abs(Ap)=abs(An), and therefore the property measure PM obtains a value of 50 in step 34. In the example of FIG. 7, the QRS wave 70 comprises a negative peak only, i.e. Ap=0, and therefore the property measure PM obtains a value of 0 in step 34.

In step 35, the value of PM is mapped to a signal attribute and the said attribute is assigned to the respective indication signal segment in that time window. It is assumed below, that perfect match (PM=100, FIG. 5) corresponds to a red indication signal color, 50 percent match (PM=50, FIG. 6) corresponds to a green indication signal color and 0 percent match (PM=0, FIG. 7) corresponds to blue indication signal color.

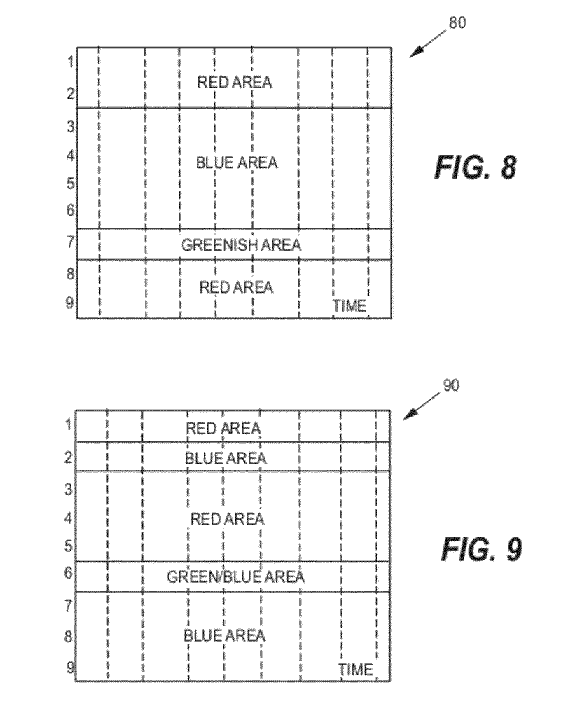

Regardless of the particular color coding used, the user display window of a normal ECG has a specific appearance or look. FIG. 8 illustrates the appearance of the user display window 80 in case of normal ECG and the above color coding. In this example, the nine ECG leads corresponding to the nine indication signal segments 1-9 are from top to bottom: I, aVF, aVR, and V1-V6. Each segment comprises consecutive “rectangles” 81 whose colors are defined by the property measures. The width of each “rectangle” is defined by the cardiac cycles of the subject, as is shown by vertical dashed lines in the figure, while the height of the rectangles corresponds to the height of the display rows. In case of a normal ECG, the user display window has a red area both at the top and at the bottom and a blue area in the middle. This is because normal ECG shows positive QRS waves in both leads I and aVF and therefore the indicator signals of the two topmost rows are red, as illustrated in FIG. 8. Furthermore, normal ECG should show negative QRS in aVR lead, i.e. blue color in the third row. Leads V1 and V2 should also show negative QRS (blue color in the 4^{th} and 5^{th} rows). Leads V5 and V6 should show positive QRS waves according to normal QRS progression in the V leads. This can be seen as red color in the 8^{th} and 9^{th} rows of the user display window. Consequently, normal ECG should produce red color at the top and bottom of the user display window and blue in the middle, as is indicated in FIG. 8.

FIG. 9 illustrates the user display window 90 for the same nine ECG lead signals 1-9 in case of a patient with abnormal ECG conduction (left anterior fascicular block (LAFB) and right bundle branch block (RBBB)). In this case, aVF is negative, which results in a blue indicator signal in the 2^{nd} row. Lead aVR, however, is positive as indicated with red color in the 3^{rd} row. Further, V1 and V2 leads show positive QRS waves as indicated by red color in the 4^{th} and 5^{th} rows. Leads V5 and V6 in turn show negative QRS waves as indicated by blue color in the 8^{th} and 9^{th} rows. As obvious from FIG. 9, any caregiver should be able to recognize that the user display window of FIG. 9 is different from a user display window of a normal ECG.

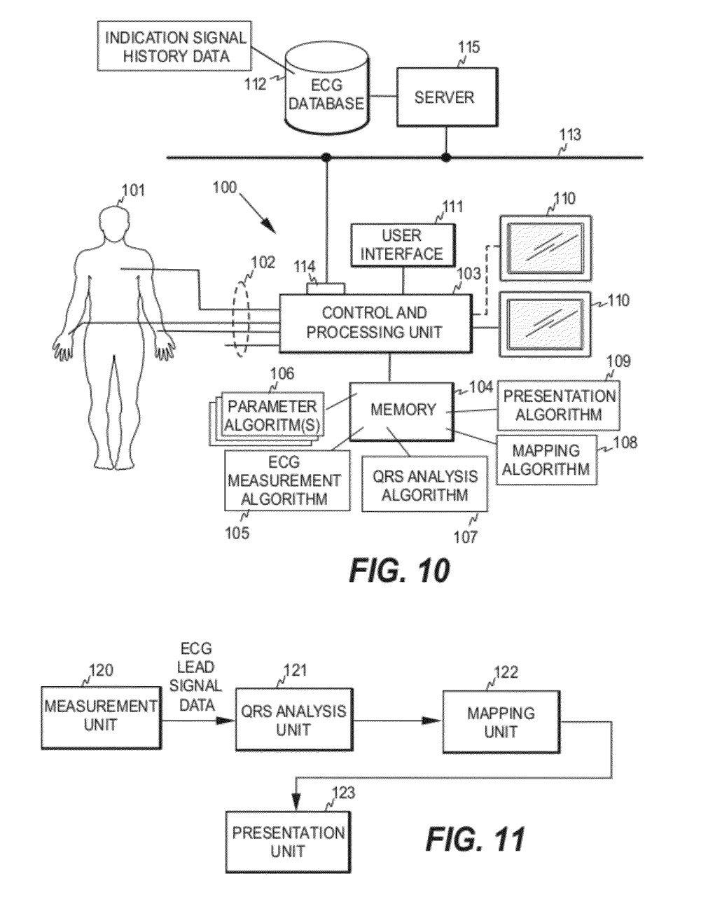

FIG. 10 illustrates one embodiment of a monitoring apparatus/system 100 for monitoring a subject 101. A monitoring apparatus/system normally acquires a plurality of physiological signals 102 from the subject, where one physiological signal corresponds to one measurement channel. The physiological signals typically comprise several types of signals, such as ECG, EEG, blood pressure, respiration, and plethysmographic signals. Based on the raw real-time physiological signal data obtained from the subject, a plurality of physiological parameters may be determined. A physiological parameter here refers to a variable calculated from the waveform data of one or more of the physiological signals acquired from the subject. If a physiological parameter is derived from more than one physiological signal, i.e. from more than one measurement channel, the said physiological signals are usually of the same signal type. The physiological parameter may thus also represent a waveform signal value determined over a predefined period of time, although the physiological parameter is typically a distinct parameter derived from one or more measurement channels, such as heart rate derived from an ECG signal or an Spa) value derived from a plethysmographic signal. Each signal parameter may be assigned one or more alarm limits to alert the nursing staff when the parameter reaches or crosses the alarm limit.

The physiological signals 102 acquired from the subject 101 are supplied to a control and processing unit 103 through a pre-processing stage (not shown) comprising typically an input amplifier and a filter, for example. The control and processing unit converts the signals into digitized format for each measurement channel. The digitized signal data may then be stored in the memory 104 of the control and processing unit.

As the disclosed measurement concerns ECG measurement, the apparatus/system is discussed in terms of the ECG measurement in this context. However, it is to be noted that no real ECG electrode placement is shown in FIG. 10. For the ECG measurement, the control and processing unit may be provided with a separate ECG measurement algorithm 105 adapted to acquire the ECG lead signal data from the subject. For the determination of the ECG related parameters, the control and processing unit may further be provided with an ECG parameter algorithm 106 adapted to calculate ECG related parameters. The control and processing unit may further be provided with a QRS analysis algorithm 107 adapted to calculate the property measures, with a mapping algorithm 108 adapted to map the property measures to attributes of the indication signals, and with a presentation algorithm 109 adapted to control a user output device, such as a display unit 110, to present the indication signals to a user of the apparatus. Operations of the ECG parameter algorithm and the QRS analysis algorithm may also be combined. For example, the operations corresponding to steps 31 and 32 may be carried out by the parameter algorithm, while the operations corresponding to steps 33 and 34 may be carried out by the QRS analysis algorithm.

Consequently, in terms of the disclosed ECG monitoring process, the functionalities of the control and processing unit 103 may be divided into the units shown in FIG. 11. A measurement unit 120 is configured to acquire the lead signal data, a QRS analysis unit 121 is configured to analyze the lead signal data and to determine the property measures, a mapping unit 122 is configured to map the property measures to the attributes of the indication signal, and a presentation unit 123 is configured to control a user output device, such as a display unit, to display the indication signal.

It is to be noted that FIGS. 10 and 11 illustrate the division of the functionalities of the control and processing unit in logical sense and in view of the visually informative ECG monitoring disclosed. In a real apparatus the functionalities may be distributed in different ways between the elements or units of the apparatus.

In the above examples, the property measure is mapped to a signal attribute similarly for all indication signals or signal segments. That is, a certain ECG property, such as the positive QRS polarity of FIG. 5, corresponds to a certain color, while negative polarity corresponds to another color. However, the color coding may also be lead-specific so that the property that is normal for a specific lead signal corresponds to a certain color in the respective indication signal or signal segment. For example, normal property may correspond to green and abnormal to red. In this way, all indication signals are green or greenish in case of a normal ECG, and abnormalities are easy to notice. Normal may here be defined in terms of general population or in terms of the patient in question or a smaller patient group to which the present patient belongs. For example, in terms of general population lead V1 is negative. However, sometimes it may be quite normal to have a Q wave in some of the leads and therefore an abnormal axis as compared to general population. This deviation from the general population applies to infarction patients, for example.

In one embodiment, a user display window of a normal ECG may be displayed as a reference window, so that the user may visually compare the colors of current user display window with those of the reference window. The reference window may be obtained from the present subject during his or her normal ECG or from a greater population with normal ECG. The monitoring of a subject may also be carried out so that the user display window, and possibly also the reference window, is/are displayed only if the ECG starts to deviate from normal ECG.

A conventional patient monitor may also be upgraded to show the colour coded user display window in addition to the conventional waveform presentation (FIGS. 1 and 2). Such an upgrade may be implemented, for example, by delivering to the monitor a plug-in unit that may be provided with the necessary software portions for enabling the control and processing unit to generate the colour coded user display window based on the ECG lead signal data. When the color coded user display window is taken into use, the control and processing unit 103 executes the software portions to display the user display window to the user of apparatus/system. These software portions may correspond to the operational units 121-123 of FIG. 11, for example. However, the contents of the software portions may vary depending on the existing algorithms of the apparatus. The plug-in unit may be delivered, for example, on a data carrier, such as a CD or a memory card, or the through a telecommunications network. The patient monitor may display both a conventional user display window according to FIGS. 1 and 2 and a user display according to FIGS. 8 and 9 in which one or more properties of the ECG lead signals are visualized through the indication signals or signal segments.

Although the above examples relate to visually informative user output signals, the indication signal may also assume other formats, such as an audio format. For example, an audio signal may be generated if the property measures are not normal within a certain time window comprising one or more cardiac cycles. The indication signal may also be a combinatory signal. For, example an audio alert may be given if the display signals meet a predetermined criterion. Currently QRS detection signal is used as an indication of an abnormal timing of the beat indicating PVC (premature ventricular contraction) beat. A more intuitive method would be to use an audio indication signal indicating abnormal beats. Normal beat could be indicated by a first audio sound, such as “beep”, and an abnormal beat by a second audio sound, such as “toot”. A change in the sequence of audio sounds would then tell the user to check the patient.

The property measure may also be indicative of other features than QRS polarity. For example, one important feature is the duration of the QRS wave, and features related to the repolarization phase may also be considered. For example, T-wave amplitude or shape may be used to indicate a T-wave change that may predict advance events, such as infarction. Some users may be interested in neonatal apnea events. Due to fact that heart rate (HR) typically decreases during those events, it may be useful to use HR as a source for the property measure. Another useful application would be to use amplitude variation of QRS waves during respiration.

Instead of mapping of each property measure to a signal attribute the signal attributes of the indication signal may also be determined through another mechanism.

With reference to FIG. 10 again, the indication signal data may be stored in the memory 104 of the apparatus or in a memory/database 112 of a network, such as a hospital LAN 113. The time scale of the user display window may be adjustable, so that a user may select a desired time period for review. The indication signal history data corresponding to the selected time period may be retrieved from the network memory 112 through a network interface 114 and a database server 115 and displayed in the user display window on the screen of the display unit 110. In this way the user of the apparatus may examine, whether a specific event has occurred earlier by searching for certain colors or color patterns from the user display window covering the desired time period. The colors also a give a clear indication of the rate of the event of interest. For example, premature ventricular complexes and their occurrence rate may be indicated intuitively in this way. The visualization of the ECG in the user display window provides a physician an efficient tool to quickly search for certain events from the ECG history of the subject, and to get an impression of the occurrence rate of the events. It is also possible that the indication signal(s) is/are used only for the examination of the patient history, by producing the said signal(s) off-line based on the lead signal data stored earlier in a memory or database.

Above, ECG is mainly used as an example of the physiological signal. However, the above mechanism may also be applied to invasive blood pressure, for example, where the variation of pulse amplitude increases during hypovolemia, which may be an indication of internal bleeding or sepsis. Indicating different types of pressure pulses to the user in the above-described manner provides a way to get an early warning of adverse developments. Similar application possibilities may be found from pulse oximetry. As implied above, the disclosed mechanism may also be applied to a waveform of a physiological parameter derived from a physiological signal, such as an SpO2 time series derived from a plethysmographic signal. Consequently, the term “physiological signal” in the appended claims covers both alternatives and may thus refer to a sequence of physiological signal or parameter values. The display may also contain different signals/parameters, such as ECG and plethysmographic signals/parameters, at the same time.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural or operational elements that do not differ from the literal language of the claims, or if they have structural or operational elements with insubstantial differences from the literal language of the claims.

1. A method for monitoring a physiological signal of a subject, the method comprising:
deriving a property measure from at least one physiological signal obtained from a subject, wherein each property measure is indicative of a predetermined property of a respective physiological signal in a time window, and wherein the deriving is performed in consecutive time windows, thereby to obtain at least one property measure sequence;
producing at least one indication signal to be presented to a user, wherein the producing comprises determining signal attributes for the at least one indication signal based on the at least one property measure sequence; and
presenting the at least one indication signal to the user.

2. The method according to claim 1, wherein the deriving comprises deriving the property measure from each of a plurality of physiological signals, in which each physiological signal is an ECG lead signal, thereby to obtain a corresponding plurality of property measure sequences.

3. The method according to claim 1, wherein the producing comprises mapping each property measure to a signal attribute, thereby to obtain at least one signal attribute in each time window.

4. The method according to claim 2, wherein the deriving is performed in consecutive time windows, in which the time windows correspond to cardiac cycles of the subject.

5. The method according to claim 4, wherein the deriving includes deriving the property measure, in which the property measure is indicative of polarity of a QRS wave in respective ECG lead signal.

6. The method according to claim 3, wherein the mapping comprises mapping each property measure to the signal attribute, in which the signal attribute is a signal color.

7. The method according to claim 2, wherein the producing includes producing a video signal comprising signal segments corresponding to the plurality ECG lead signals and the determining includes controlling colors of each signal segment according to respective signal attribute sequence.

8. The method according to claim 7, wherein the presenting comprises presenting the signal segments aligned with respect to each other on a screen of a display unit.

9. The method according to claim 1, wherein the producing includes producing the at least one indication signal, in which the at least one indication signal includes an audio signal.

10. An apparatus for monitoring a physiological signal of a subject, the apparatus comprising:
an analysis unit adapted to derive a property measure from at least one physiological signal obtained from a subject, wherein each property measure is indicative of a predetermined property of a respective physiological signal in a time window, and wherein the analysis unit is adapted to derive the property measure in consecutive time windows, thereby to obtain at least one property measure sequence; and
a presentation unit adapted to determine signal attributes for at least one indication signal based on the at least one property measure sequence, produce the at least one indication signal and present the at least one indication signal to a user.

11. The apparatus according to claim 10, wherein the at least one physiological signal comprises a plurality of ECG lead signals, thereby to obtain a corresponding plurality of property measure sequences.

12. The apparatus according to claim 10, wherein the presentation unit comprises a mapping unit adapted to map each property measure to a signal attribute, thereby to obtain at least one signal attribute in each time window.

13. The apparatus according to claim 11, wherein the time windows correspond to cardiac cycles of the subject.

14. The apparatus according to claim 13, wherein the property measure is indicative of polarity of a QRS wave in respective ECG lead signal.

15. The apparatus according to claim 12, wherein the signal attribute is a signal color.

16. The apparatus according to claim 11, wherein the presentation unit is configured to produce a video signal comprising signal segments corresponding to the plurality of ECG lead signals and to control colors of each signal segment according to respective signal attribute sequence.

17. The apparatus according to claim 16, wherein the presentation unit is adapted to present the signal segments vertically aligned one below the other on a screen of a display unit.

18. The apparatus according to claim 10, wherein the at least one indication signal includes an audio signal.

19. A computer program product for monitoring a physiological signal of a subject, the computer program product comprising:
a first program product portion adapted to derive a property measure from at least one physiological signal obtained from a subject, wherein each property measure is indicative of a predetermined property of a respective physiological signal in a time window, and wherein the first program product portion is adapted to derive the property measure in consecutive time windows, thereby to obtain at least one property measure sequence; and
a second program product portion adapted to determine signal attributes for at least one indication signal based on the at least one property measure sequence, produce the at least one indication signal, and present the at least one indication signal to a user.
